(19) United States
(12) Patent Application Publication
(10) Pub. No.: US 2019/0378431 A1
(43) Pub. Date: Dec. 12, 2019

(54) Leveraging Wearable Sensors for Context-Aware Personalized Recommendations

(71) Applicant: International Business Machines Corporation, Armonk, NY (US)

(72) Inventors: Vijay Ekambaram, Chennai (IN); Ramasuri Narayanam, Andhra Pradesh (IN); Yedendra Shrinivasan, New York, NY (US)

(21) Appl. No.: 16/003,597

(22) Filed: Jun. 8, 2018

(51) Int. Cl. G09B 19/00; G16H 40/63; G16H 20/30; G16H 20/60; A63B 24/00; G09B 5/02

(52) U.S. Cl. CPC G09B 19/00; G16H 40/63; G16H 20/30; G06Q 40/08; A63B 24/0062; G09B 5/02; G16H 20/60

(57) ABSTRACT

Methods, systems, and computer program products for leveraging wearable sensors for context-aware personalized recommendations are provided herein. A computer-implemented method includes generating, based on user-provided information, health-related recommendations to the user; monitoring, based on data derived from wearable sensors worn by the user, user activity; determining deviation information related to deviations from the health-related recommendations by the user, based the monitored activity; determining user-specific context information based on the user-provided information and/or information derived from additional sources; and generating, based on the deviation information and the user-specific context information, (i) additional health-related recommendations and (ii) incentives related to carrying out the additional health-related recommendations.

### FIELD

The present application generally relates to information technology, and, more particularly, to user health-related technologies.

### BACKGROUND

Costs related to healthcare have been increasing over time, and such increases are commonly assumed by insurance providers and/or passed on to consumers. Accordingly, recommending appropriate user/consumer actions based on the users' health conditions and other factors is of increasing importance. However, users may not follow such recommendations due to various preferences, which creates a number of additional challenges. Consequently, healthcare professionals and/or insurance providers would benefit from an ability to measure the extent to which users follow recommended actions. Nevertheless, existing healthcare management approaches fail to provide personalized techniques that take into account the context and consequence of user deviations from recommended actions.

### SUMMARY

In one embodiment of the present invention, techniques for leveraging wearable sensors for context-aware personalized recommendations are provided. An exemplary computer-implemented method can include generating, based at least in part on user-provided information, one or more health-related recommendations to the user, and monitoring, based at least in part on data derived from one or more wearable sensors worn by the user, user activity. Such a method can also include determining deviation information related to one or more deviations from the one or more health-related recommendations by the user, based at least in part on a comparison of the monitored activity to the one or more health-related recommendations, and determining one or more items of user-specific context information based at least in part on one or more of the user-provided information and information derived from one or more additional sources. Further, such a method can additionally include generating, based at least in part on the deviation information and the one or more items of user-specific context information, (i) one or more additional health-related recommendations to the user and (ii) one or more incentives related to carrying out the one or more additional health-related recommendations.

In another embodiment of the invention, an exemplary computer-implemented method can include generating, based at least in part on the deviation information and the one or more items of user-specific context information, (i) one or more additional health-related recommendations to the user, (ii) one or more incentives related to carrying out the one or more additional health-related recommendations, and (iii) one or more penalties related to failing to carry out the one or more additional health-related recommendations. Such a method can also include generating a user score based at least in part on comparing, for the user to that of one or more previous users, (i) the user-provided information, (ii) the deviation information, (iii) the one or more additional health-related recommendations, (iv) the one or more incentives related to carrying out the one or more additional health-related recommendations, and (v) the one or more penalties related to failing to carry out the one or more additional health-related recommendations.

Another embodiment of the invention or elements thereof can be implemented in the form of a computer program product tangibly embodying computer readable instructions which, when implemented, cause a computer to carry out a plurality of method steps, as described herein. Furthermore, another embodiment of the invention or elements thereof can be implemented in the form of a system including a memory and at least one processor that is coupled to the memory and configured to perform noted method steps. Yet further, another embodiment of the invention or elements thereof can be implemented in the form of means for carrying out the method steps described herein, or elements thereof; the means can include hardware module(s) or a combination of hardware and software modules, wherein the software modules are stored in a tangible computer-readable storage medium (or multiple such media).

These and other objects, features and advantages of the present invention will become apparent from the following detailed description of illustrative embodiments thereof, which is to be read in connection with the accompanying drawings.

### BRIEF DESCRIPTION OF THE DRAWINGS

FIG. 1 is a diagram illustrating system architecture and data flow, according to an embodiment of the invention;

FIG. 2 is a diagram illustrating pattern matching, according to an exemplary embodiment of the invention;

FIG. 3 is a flow diagram illustrating techniques according to an embodiment of the invention;

FIG. 4 is a system diagram of an exemplary computer system on which at least one embodiment of the invention can be implemented;

FIG. 5 depicts a cloud computing environment according to an embodiment of the present invention; and

FIG. 6 depicts abstraction model layers according to an embodiment of the present invention.

### DETAILED DESCRIPTION

As described herein, an embodiment of the present invention includes leveraging wearable sensors for context-aware personalized recommendations. At least one embodiment of the invention includes generating health-related recommendations for a user and detecting the degree and/or type of one or more user-deviations from the recommendations considering the user's profile and context. Additionally, such an embodiment can also include cognitively deriving and recommending user context-specific penalties and/or incentives. Also, one or more embodiments can include predicting one or more penalties for a given period of time (for example, the next month or year) for one or more specific types of deviations, and estimating a score for the user based on personalized cost-consequences for the degree(s) and type(s) of deviations.

At least one embodiment of the invention can include gathering context and consequence data with personalized information for one or more users. Such data can be subsequently used to generate cognitive decisions and/or recommendations pertaining to one or more health-related topics and a given user. By way merely of example, suppose a recommendation for a user includes exercising for one hour every day. If the user has recently undergone a surgery and/or experienced an accident, it may not make sense for him or her to exercise (or exercise for an hour, or exercise every day). In one or more embodiments of the invention, such context information (that is, that the user has recently undergone a surgery and/or experienced an accident), is considered as an input in generating health-related user recommendations and calculating user incentives (such as insurance premium discounts, etc.).

By way of yet another example, suppose a recommendation calls for the user to take a walk for 45 minutes every morning. However, if it rains in the morning (or there is other inclement weather), it may make sense for the user to avoid the morning walk. In such a scenario, the weather information represents context data that can be captured and utilized by one or more embodiments of the invention.

Also, by way of additional example, consider a scenario wherein it is recommended that two people undertake 30 minutes of exercise every day, and wherein the first person is healthier than the second person. Accordingly, while both people can potentially (assuming each person follows the recommendation) undertake approximately the same amount of exercise over a given period of time, the effect of such exercise will likely be different for the two people. In such a scenario, the variable consequences of a recommendation relative to specific users can represent context data that can be captured and utilized by one or more embodiments of the invention.

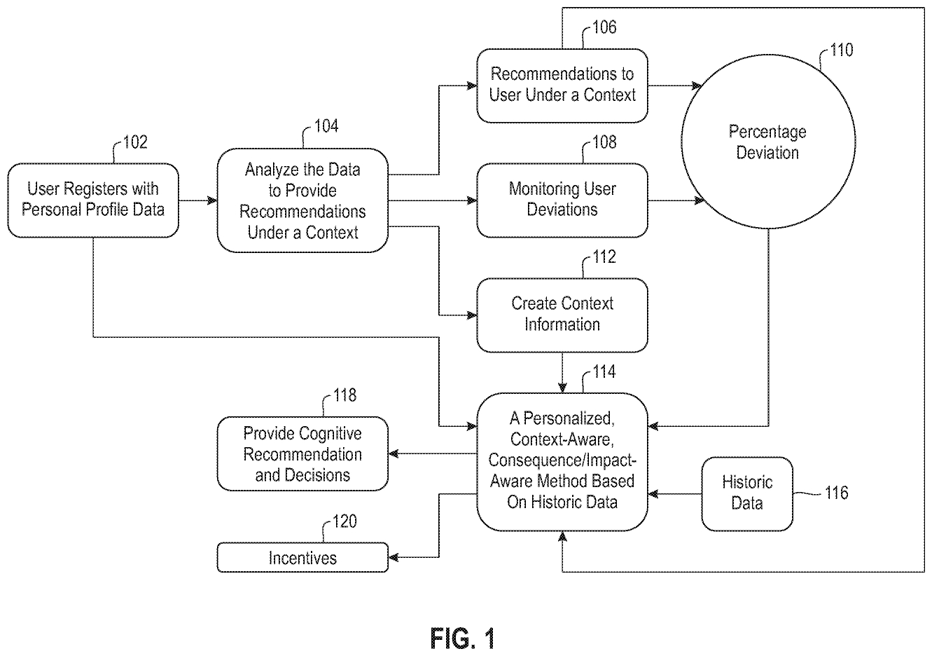

FIG. 1 is a diagram illustrating system architecture and data flow, according to an embodiment of the invention. By way of illustration, step 102 includes a user registering (for example, with a mobile software application) with profile information. Such profile information can include, for example, health-related information such as existing conditions, age, gender, medical test reports, sleep habits, exercise habits, nutrition habits, etc. Step 104 includes analyzing the data provided in step 102, wherein such analysis is to be used in providing recommendations to the user. Additionally, step 106 includes generating, and outputting to the user, one or more recommendations under a given user-related context. Further, step 108 includes monitoring one or more user activities as well as any user deviations from the one or more generated recommendations. Based on step 106 and step 108, at least one embodiment of the invention can include calculating a percentage deviation 110 from one or more of the generated recommendations by the user.

In one or more embodiments of the invention, based on the user profile information, a software application can provide one or more recommendations to the user (via a version of the application installed on one or more user devices). The software application can also include capabilities to monitor user actions such as, for example, user exercise time, user caloric intake, user calorie burn, user stress level, user sleep patterns, user eating habits, etc. In one or more embodiments of the invention, the software application can be based on one or more Internet of Things (IoT) systems (such as, for example, home and/or wearable sensors), which collect data pertaining to a user for monitoring.

Further, step 112 includes creating context information based on the analysis carried out in step 104. For example, based on the user profile information, at least one embodiment of the invention can include determining various forms of context information (such as, for example, recent health-related developments pertaining to the user (a recent injury, a recent medical procedure, etc.), weather information, variable consequences of a recommendation relative to specific users and/or user characteristics, etc.). The created context information, as well as the user profile data, the one or more generated recommendations, and the calculated percentage deviation(s), can be provided as inputs to a personalized, context-aware, consequence/impact-aware method 114, which can be derived based at least in part on historic data 116 (such as, for example, health-related data including user medical history, user allergies, user behaviors, etc.).

The method 114, based on the noted inputs, can provide one or more cognitive recommendations and/or decisions pertaining to the user in step 118, as well as provide one or more incentives (such as insurance premium discounts, etc.) to the user in step 120. With respect to providing incentives, an example embodiment of the invention can include an insurance company providing monetary incentives (such as premium discounts) to a user based at least in part on the user's current premium, the expected expenditure associated with the user, and an expected margin related thereto. Additionally, such a cognitive recommendation and/or decision can include a revised recommendation (that is, revised in relation to the generated recommendation(s) in step 106) based on analysis of the above-noted inputs.

By way merely of illustration, consider an example scenario wherein a recommendation is generated for a user to perform exercise for one hour every day. Using inputs such as the created context information, the user profile data, and the calculated percentage deviation(s), method 114 can determine that the user has recently undergone a surgery and/or accident that would make it difficult to successfully carry out the recommendation. In such a scenario, it may not be useful and/or effective to penalize the user for failing to carry out the recommendation, and as such, the method 114 can generate (and output to the user) a new and/or revised recommendation (such as to avoid exercise for a proscribed period of time (relevant to recovery form the recent surgery and/or accident).

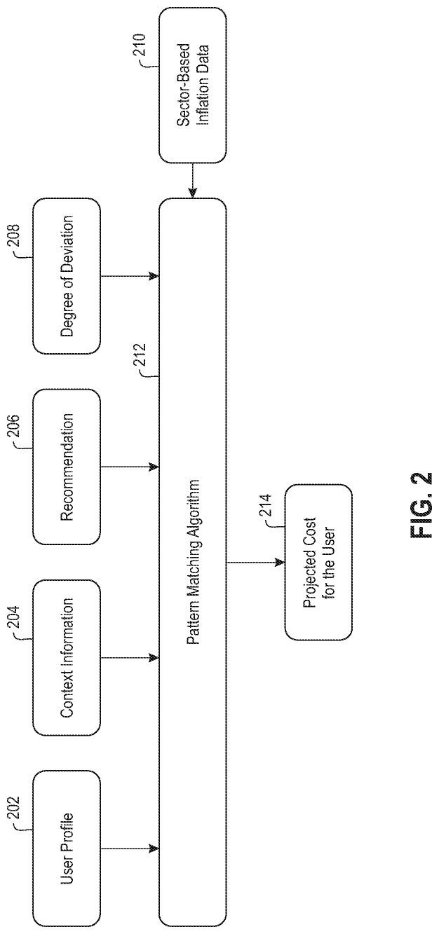

FIG. 2 is a diagram illustrating pattern matching, according to an exemplary embodiment of the invention. By way of illustration, FIG. 2 depicts a user profile 202, context information 204, a recommendation 206, a degree of deviation 208 from the recommendations 206 by the user, and sector-based inflation data 210, which can all be provided as input to a pattern matching algorithm 212. In connection with the above-noted sector-based inflation data 210, a sector refers to one or more geographic areas of users used as a parameter for analysis. The pattern matching algorithm 212 can include historical cost consequence data for deviations from one or more recommended actions for given user profiles and context information, and the pattern matching algorithm 212 can use such data, in conjunction with the above-noted inputs, to generate a pattern match with respect to a current user. In at least one embodiment of the invention, the pattern matching algorithm 212 learns one or more correlations between components 202, 204, 206, 208, and 210, and generates a prediction 214 for new user data (given components 202, 204, 206, 208 and 210).

As also depicted in FIG. 2, the pattern matching algorithm 212 can generate and output a projected cost for the user. Such a projected cost can be represented in the form of an estimated health score for the user based on personalized cost-consequences for the degree and type of deviation(s) captured as inputs to the pattern matching algorithm 212. At least one embodiment of the invention can include crowd-sourcing health-related data, personalized cost-consequences for the degree and type of deviations made, risks underwent, etc. Such an embodiment can additionally include applying the (unsupervised) pattern matching algorithm 212 to such data to discover one or more patterns between user risk (related to health-related issues) and the personalized cost-consequences for the degree and type of deviations made. Based on the results thereof, such an embodiment can include estimating a health score for the one or more users in question.

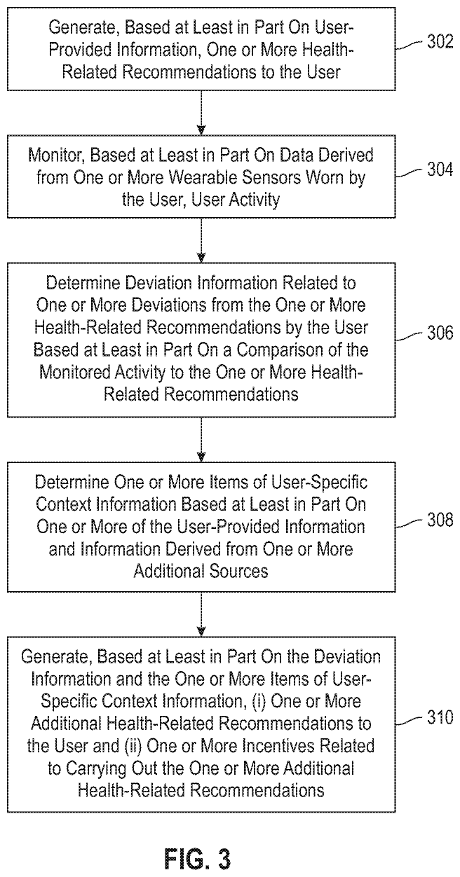

FIG. 3 is a flow diagram illustrating techniques according to an embodiment of the present invention. Step 302 includes generating, based at least in part on user-provided information, one or more health-related recommendations to the user. The user-provided information can include one or more existing conditions, user age, user gender, one or more medical test reports, one or more user sleep habits, one or more user exercise habits, and/or one or more user nutrition habits.

Step 304 includes monitoring, based at least in part on data derived from one or more wearable sensors worn by the user, user activity. Monitoring the user activity can include monitoring one or more user exercise habits, user caloric intake, user stress level, and/or one or more user sleep patterns.

Step 306 includes determining deviation information related to one or more deviations from the one or more health-related recommendations by the user, based at least in part on a comparison of the monitored activity to the one or more health-related recommendations. The deviation information can include a degree of deviation from the one or more health-related recommendations by the user and/or a type of deviation from the one or more health-related recommendations by the user.

Step 308 includes determining one or more items of user-specific context information based at least in part on one or more of the user-provided information and information derived from one or more additional sources. The one or more items of user-specific context information can include one or more recent health-related developments pertaining to the user, current weather information, forecasted weather information, and/or information detailing variable consequences of a recommendation relative to multiple users.

Step 310 includes generating, based at least in part on the deviation information and the one or more items of user-specific context information, (i) one or more additional health-related recommendations to the user and (ii) one or more incentives related to carrying out the one or more additional health-related recommendations. The techniques depicted in FIG. 3 can also include determining one or more cost-consequences for each deviation from the one or more health-related recommendations by the user, wherein this determination can be based at least in part on (i) a type of deviation attributed to each deviation, (ii) a degree of deviation attributed to each deviation, (iii) the user-provided information, and (iv) the one or more items of user-specific context information.

Also, an additional embodiment of the invention includes generating, based at least in part on the deviation information and the one or more items of user-specific context information, (i) one or more additional health-related recommendations to the user, (ii) one or more incentives related to carrying out the one or more additional health-related recommendations, and (iii) one or more penalties related to failing to carry out the one or more additional health-related recommendations. Such an embodiment can also include generating a user score based at least in part on comparing, for the user to that of one or more previous users, (i) the user-provided information, (ii) the deviation information, (iii) the one or more additional health-related recommendations, (iv) the one or more incentives related to carrying out the one or more additional health-related recommendations, and (v) the one or more penalties related to failing to carry out the one or more additional health-related recommendations.

The techniques depicted in FIG. 3 can also, as described herein, include providing a system, wherein the system includes distinct software modules, each of the distinct software modules being embodied on a tangible computer-readable recordable storage medium. All of the modules (or any subset thereof) can be on the same medium, or each can be on a different medium, for example. The modules can include any or all of the components shown in the figures and/or described herein. In an embodiment of the invention, the modules can run, for example, on a hardware processor. The method steps can then be carried out using the distinct software modules of the system, as described above, executing on a hardware processor. Further, a computer program product can include a tangible computer-readable recordable storage medium with code adapted to be executed to carry out at least one method step described herein, including the provision of the system with the distinct software modules.

Additionally, the techniques depicted in FIG. 3 can be implemented via a computer program product that can include computer useable program code that is stored in a computer readable storage medium in a data processing system, and wherein the computer useable program code was downloaded over a network from a remote data processing system. Also, in an embodiment of the invention, the computer program product can include computer useable program code that is stored in a computer readable storage medium in a server data processing system, and wherein the computer useable program code is downloaded over a network to a remote data processing system for use in a computer readable storage medium with the remote system.

An embodiment of the invention or elements thereof can be implemented in the form of an apparatus including a memory and at least one processor that is coupled to the memory and configured to perform exemplary method steps.

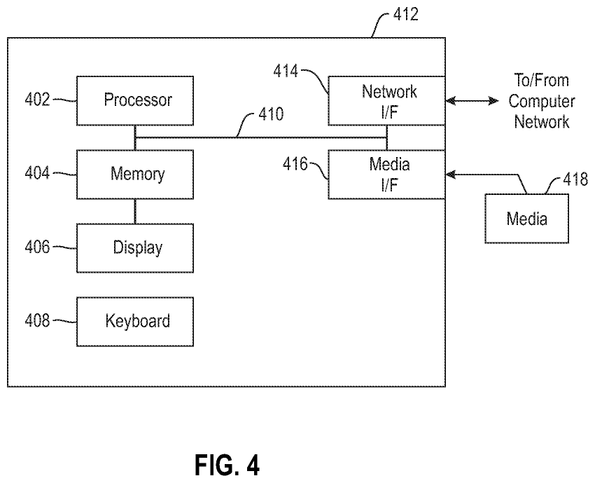

Additionally, an embodiment of the present invention can make use of software running on a computer or workstation. With reference to FIG. 4, such an implementation might employ, for example, a processor 402, a memory 404, and an input/output interface formed, for example, by a display 406 and a keyboard 408. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory), ROM (read only memory), a fixed memory device (for example, hard drive), a removable memory device (for example, diskette), a flash memory and the like. In addition, the phrase “input/output interface” as used herein, is intended to include, for example, a mechanism for inputting data to the processing unit (for example, mouse), and a mechanism for providing results associated with the processing unit (for example, printer). The processor 402, memory 404, and input/output interface such as display 406 and keyboard 408 can be interconnected, for example, via bus 410 as part of a data processing unit 412. Suitable interconnections, for example via bus 410, can also be provided to a network interface 414, such as a network card, which can be provided to interface with a computer network, and to a media interface 416, such as a diskette or CD-ROM drive, which can be provided to interface with media 418.

Accordingly, computer software including instructions or code for performing the methodologies of the invention, as described herein, may be stored in associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 402 coupled directly or indirectly to memory elements 404 through a system bus 410. The memory elements can include local memory employed during actual implementation of the program code, bulk storage, and cache memories which provide temporary storage of at least some program code in order to reduce the number of times code must be retrieved from bulk storage during implementation.

Input/output or I/O devices (including, but not limited to, keyboards 408, displays 406, pointing devices, and the like) can be coupled to the system either directly (such as via bus 410) or through intervening I/O controllers (omitted for clarity).

Network adapters such as network interface 414 may also be coupled to the system to enable the data processing system to become coupled to other data processing systems or remote printers or storage devices through intervening private or public networks. Modems, cable modems and Ethernet cards are just a few of the currently available types of network adapters.

As used herein, including the claims, a “server” includes a physical data processing system (for example, system 412 as shown in FIG. 4) running a server program. It will be understood that such a physical server may or may not include a display and keyboard.

The present invention may be a system, a method, and/or a computer program product at any possible technical detail level of integration. The computer program product may include a computer readable storage medium (or media) having computer readable program instructions thereon for causing a processor to carry out embodiments of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform embodiments of the present invention.

Embodiments of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It should be noted that any of the methods described herein can include an additional step of providing a system comprising distinct software modules embodied on a computer readable storage medium; the modules can include, for example, any or all of the components detailed herein. The method steps can then be carried out using the distinct software modules and/or sub-modules of the system, as described above, executing on a hardware processor 402. Further, a computer program product can include a computer-readable storage medium with code adapted to be implemented to carry out at least one method step described herein, including the provision of the system with the distinct software modules.

In any case, it should be understood that the components illustrated herein may be implemented in various forms of hardware, software, or combinations thereof, for example, application specific integrated circuit(s) (ASICS), functional circuitry, an appropriately programmed digital computer with associated memory, and the like. Given the teachings of the invention provided herein, one of ordinary skill in the related art will be able to contemplate other implementations of the components of the invention.

Additionally, it is understood in advance that implementation of the teachings recited herein are not limited to a particular computing environment. Rather, embodiments of the present invention are capable of being implemented in conjunction with any type of computing environment now known or later developed.

For example, cloud computing is a model of service delivery for enabling convenient, on-demand network access to a shared pool of configurable computing resources (for example, networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (for example, country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (for example, storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (for example, web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (for example, host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (for example, mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (for example, cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

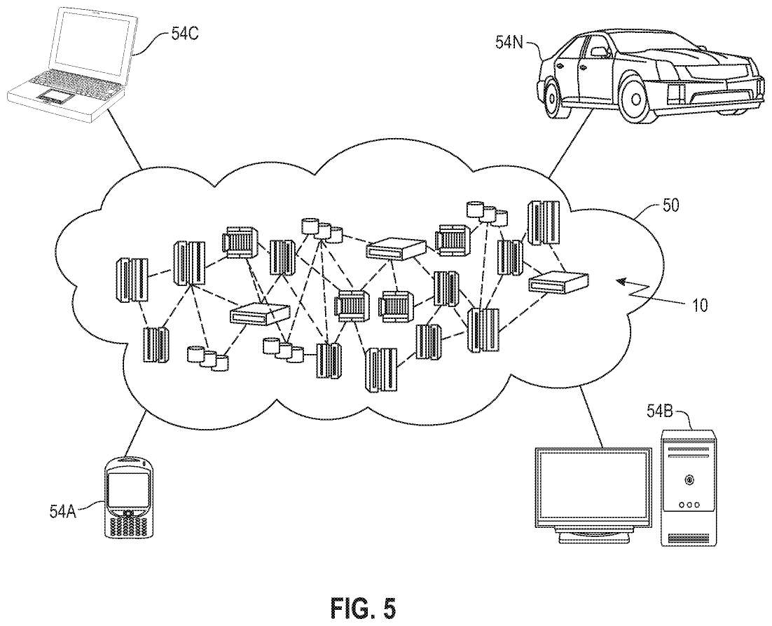

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

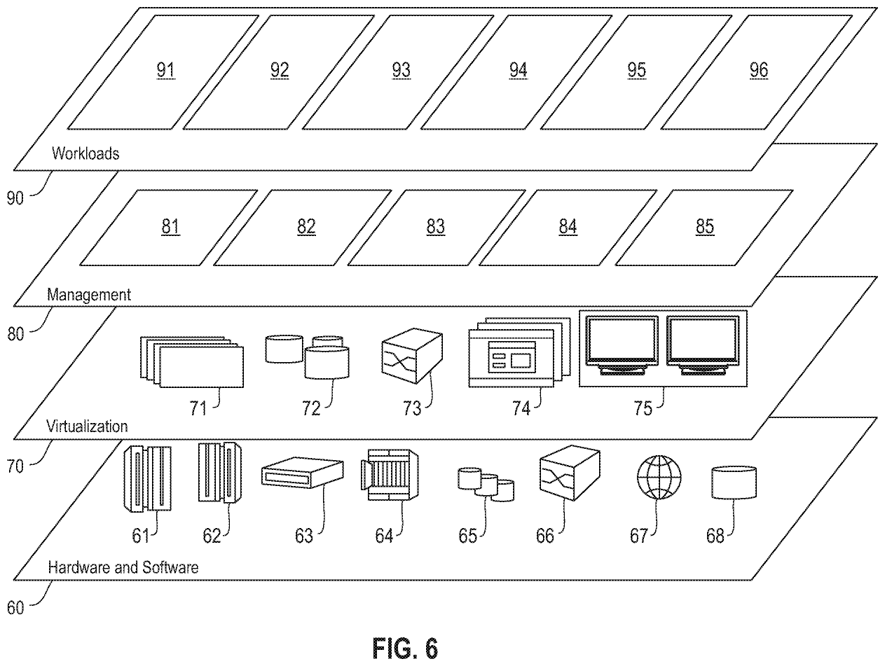

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75. In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources.

In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and context-aware personalized recommendation generation 96, in accordance with the one or more embodiments of the present invention.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of another feature, step, operation, element, component, and/or group thereof.

At least one embodiment of the present invention may provide a beneficial effect such as, for example, leveraging wearable sensors to generate context-aware personalized health-related recommendations.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method, the method comprising steps of:
generating, based at least in part on user-provided information, one or more health-related recommendations to the user;
monitoring, based at least in part on data derived from one or more wearable sensors worn by the user, user activity;
determining deviation information related to one or more deviations from the one or more health-related recommendations by the user, based at least in part on a comparison of the monitored activity to the one or more health-related recommendations;
determining one or more items of user-specific context information based at least in part on one or more of (i) the user-provided information and (ii) information derived from one or more additional sources; and
generating, based at least in part on the deviation information and the one or more items of user-specific context information, (i) one or more additional health-related recommendations to the user and (ii) one or more incentives related to carrying out the one or more additional health-related recommendations;
wherein the steps are carried out by at least one computing device.

2. The computer-implemented method of claim 1, wherein the user-provided information comprises at least one of (i) one or more existing conditions, (ii) user age, (iii) user gender, (iv) one or more medical test reports, (v) one or more user sleep habits, (vi) one or more user exercise habits, and (vii) one or more user nutrition habits.

3. The computer-implemented method of claim 1, wherein said monitoring the user activity comprises monitoring one or more user exercise habits.

4. The computer-implemented method of claim 1, wherein said monitoring the user activity comprises monitoring user caloric intake.

5. The computer-implemented method of claim 1, wherein said monitoring the user activity comprises monitoring user stress level.

6. The computer-implemented method of claim 1, wherein said monitoring the user activity comprises monitoring one or more user sleep patterns.

7. The computer-implemented method of claim 1, wherein the one or more items of user-specific context information comprises one or more recent health-related developments pertaining to the user.

8. The computer-implemented method of claim 1, wherein the one or more items of user-specific context information comprises current weather information.

9. The computer-implemented method of claim 1, wherein the one or more items of user-specific context information comprises forecasted weather information.

10. The computer-implemented method of claim 1, wherein the one or more items of user-specific context information comprises information detailing variable consequences of a recommendation relative to multiple users.

11. The computer-implemented method of claim 1, comprising:
determining one or more cost-consequences for each deviation from the one or more health-related recommendations by the user, wherein said determining is based at least in part on (i) a degree of deviation attributed to each deviation, (ii) the user-provided information, and (iii) the one or more items of user-specific context information.

12. A computer program product comprising a computer readable storage medium having program instructions embodied therewith, the program instructions executable by a computing device to cause the computing device to:
generate, based at least in part on user-provided information, one or more health-related recommendations to the user;
monitor, based at least in part on data derived from one or more wearable sensors worn by the user, user activity;
determine deviation information related to one or more deviations from the one or more health-related recommendations by the user, based at least in part on a comparison of the monitored activity to the one or more health-related recommendations;
determine one or more items of user-specific context information based at least in part on one or more of (i) the user-provided information and (ii) information derived from one or more additional sources; and
generate, based at least in part on the deviation information and the one or more items of user-specific context information, (i) one or more additional health-related recommendations to the user and (ii) one or more incentives related to carrying out the one or more additional health-related recommendations.

13. The computer program product of claim 12, wherein said monitoring the user activity comprises monitoring at least one of (i) one or more user exercise habits, (ii) user caloric intake, (iii) user stress level, and (iv) one or more user sleep patterns.

14. The computer program product of claim 12, wherein the one or more items of user-specific context information comprises at least one of (i) one or more recent health-related developments pertaining to the user, (ii) current weather information, (iii) forecasted weather information, (iv) information detailing variable consequences of a recommendation relative to multiple users.

15. A system comprising:
a memory; and
at least one processor operably coupled to the memory and configured for:
generating, based at least in part on user-provided information, one or more health-related recommendations to the user;
monitoring, based at least in part on data derived from one or more wearable sensors worn by the user, user activity;
determining deviation information related to one or more deviations from the one or more health-related recommendations by the user, based at least in part on a comparison of the monitored activity to the one or more health-related recommendations;
determining one or more items of user-specific context information based at least in part on one or more of (i) the user-provided information and (ii) information derived from one or more additional sources; and
generating, based at least in part on the deviation information and the one or more items of user-specific context information, (i) one or more additional health-related recommendations to the user and (ii) one or more incentives related to carrying out the one or more additional health-related recommendations.

16. A computer-implemented method, the method comprising steps of:
generating, based at least in part on user-provided information, one or more health-related recommendations to the user;
monitoring, based at least in part on data derived from one or more wearable sensors worn by the user, user activity;
determining deviation information related to one or more deviations from the one or more health-related recommendations by the user, based at least in part on a comparison of the monitored activity to the one or more health-related recommendations;
determining one or more items of user-specific context information, based at least in part on one or more of (i) the user-provided information and (ii) information derived from one or more additional sources;
generating, based at least in part on the deviation information and the one or more items of user-specific context information, (i) one or more additional health-related recommendations to the user, (ii) one or more incentives related to carrying out the one or more additional health-related recommendations, and (iii) one or more penalties related to failing to carry out the one or more additional health-related recommendations; and
generating a user score based at least in part on comparing, for the user to that of one or more previous users, (i) the user-provided information, (ii) the deviation information, (iii) the one or more additional health-related recommendations, (iv) the one or more incentives related to carrying out the one or more additional health-related recommendations, and (v) the one or more penalties related to failing to carry out the one or more additional health-related recommendations;
wherein the steps are carried out by at least one computing device.

17. The computer-implemented method of claim 16, wherein said monitoring the user activity comprises monitoring one or more user exercise habits.

18. The computer-implemented method of claim 16, wherein said monitoring the user activity comprises monitoring user caloric intake.

19. The computer-implemented method of claim 16, wherein said monitoring the user activity comprises monitoring one or more user sleep patterns.

20. The computer-implemented method of claim 16, wherein the one or more items of user-specific context information comprises at least one of (i) one or more recent health-related developments pertaining to the user, (ii) current weather information, (iii) forecasted weather information, (iv) information detailing variable consequences of a recommendation relative to multiple users.
